(19) United States
(12) Patent Application Publication
(10) Pub. No.: US 2003/0055075 A1
(43) Pub. Date: Mar. 20, 2003

(54) Programmable controlled release injectable opioid formulation

(76) Inventor: Reid M. Rubsamen, Alamo, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/195,046

(22) Filed: Jul. 12, 2002

Related U.S. Application Data
(60) Provisional application No. 60/305,364, filed on Jul. 13, 2001.
(60) Provisional application No. 60/326,675, filed on Oct. 2, 2001.

(51) Int. Cl. A61K 31/485; A61K 9/14; A61K 9/16; A61K 9/50

(52) U.S. Cl. 514/282; 424/489

(57) ABSTRACT

Controlled release formulations of drugs such as fentanyl and sufentanil are disclosed. The formulations are comprised of two or more different groups of particles wherein the particles of a given group are substantially identical but are different from the particles in any other group. The combined effect of the groups provides steady state blood levels which are particularly useful when administering opioids compound such as fentanyl by injection. A method of reducing unwanted diversion of narcotics is also disclosed.

### CROSS-REFERENCES

This application is the conversion of provisional applications Nos. 60/305,364 filed Jul. 13, 2001 and 60/326,675 filed Oct. 2, 2001 to which applications is claimed priority and which applications are incorporated herein by reference.

### FIELD OF THE INVENTION

The invention relates generally to drug delivery technology and more particularly to formulations of spherical particles designed to provide a particular release profile on administration. Further, the invention relates to a method of reducing unwanted diversion of narcotics away from the desired patient.

### BACKGROUND OF THE INVENTION

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous).

U.S. Pat. No. 3,773,919 describes creating slow release formulations producing a steady release of drug in the bloodstream by employing polylactide-drug mixtures in the dosage form. The inventors describe using a chemical based microencapsulation procedure for forming precipitates of the polylactide-drug mixtures suitable for injection. They discuss many potential applications for their invention including the administration of morphine.

U.S. Pat. No. 4,942,035 describes using PLGA polymer as an excipient allowing formulations to be created to facilitate the controlled release of polypeptide active drugs into solutions.

U.S. Pat. No. 5,514,380 describes modifying the cross-linking in PLGA polymer in order to obtain more controllable release profiles.

U.S. Pat. No. 5,543,158 describes potential benefits of using PLGA polymer with pharmaceutically active drug to create particles in a very small size range to minimize incorporation of the injected formulation into the patient's macrophages which would result in inactivation of the drug.

U.S. Pat. No. 5,650,173 describes an emulsion system for creating particles of PGLA and active drug suitable for injection.

U.S. Pat. No. 5,654,008 describes a technique for combining PLGA and active drug into microparticles suitable for injection by using an emulsion system created using a static mixer.

U.S. Pat. No. 5,759,583 describes using a quaternary ammonium surfactant as an excipient to facilitate the creation of PLGA drug combinations suitable for injection to create a controlled release formulation.

U.S. Pat. No. 5,912,015 describes using metal cations as release modulators in the injectable drug formulation comprising PLGA and active drug.

U.S. Pat. No. 5,916,598 describes using emulsion systems and solvent extraction techniques as tools for creating microparticles comprised of PLGA and active drug for sustained release formulations.

U.S. Pat. No. 6,254,890 describes using PLGA to create sustained release formulations containing nucleic acids.

Previous approaches for combining PLGA with active drug to create such controlled release formulations relied on chemical techniques for creating microparticles suitable for injection. These techniques have focused on the use of solvent systems to produce emulsions resulting in the creation of a precipitate of crystalline microparticle in an approximate size range suitable for injection. Other systems involve removing solvents used during the fabrication process. The US FDA as well as international drug regulatory authorities have drafted regulations strictly limiting the amount of residual solvent acceptable in marketed pharmaceutical preparations (ICH Harmonized Tripartite Guideline Q3C Impurities: “Guidelines for Residual Solvents”).

Additional discussion of categories of systems for controlled release may be found in Agis F. Kydonieus, Controlled Release Technologies: Methods, Theory and Applications, 1980 (CRC Press, Inc.).

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems is “preprogrammed” at specific rate profiles. This may be accomplished by system design, which controls the molecular diffusion of drug molecules in and/or across the barrier medium within or surrounding the delivery system. Fick's laws of diffusion are often followed.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

Another controlled release dosage form is a complex between an ion exchange resin and the lipoates. Ion exchange resin-drug complexes have been used to formulate sustained-release products of acidic and basic drugs. In one preferable embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable micro spheres are another controlled release dosage form. Injectable micro spheres may be prepared by non-aqueous phase separation techniques, and spray-drying techniques. Micro spheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm.

Bull. 35:1538-1545 (1987).

### SUMMARY OF THE INVENTION

A formulation of different groups of spherical particles is disclosed. Each group of spherical particles consists of multiple particles which are all substantially the same size which together with other groups are designed to provide a combination of different drug release rates on administration and provide a relatively constant blood level of drug to the patient. The different groups of particles are formulated together to obtain a desired drug release profile. As the release rate of one group is decreasing (or the drug released from the group is being metabolized out of the system) the release rate of another group is increasing (or drug from one group is being added to the system) so that the combined groups of the formulation provide a substantially constant level of drug over a therapeutically effective period of time.

The methodology described here substantially reduces the trial and error of producing a controlled release formulation. This is done by using particles of a known size (volume and surface areas ±10%) shape (spherical) and dissolution rate within an environment to which the particles are delivered. Because all the particles of any given group have substantially the same surface area from one particle to another the dissolution rate of a given particle and the group of particles can be calculated mathematically based on a known dissolution rate of a particle of known surface area. Particles in the formulation preferably have an inner core diameter in a range of from about 1 micron to about 20 microns. The particle types may include particles comprised of drug without any coating. However, a formulation preferably comprises particles of different types wherein each different type is comprised of a different thickness of coating material surrounding and uniformly encapsulating a spherical core of pharmaceutically active drug which may be pure drug or drug combined with excipient.

An aspect of this invention is to show that in addition to relying on the chemical properties of injected microparticles for their controlled release characteristics, the physical size of these particles can be used to provide another layer of control over the release profile because that the physical size of particles in different groups of particles can be controlled precisely as can the total surface area of all the particles in the group combined. When the particles are very small in size (e.g. 1-20 micrometers) the surface area differential from one group to the next can be made quite large by small changes in diameter.

It is another aspect of this invention to show that poly (lactide-co-glycolide) polymers (PLGA) can be used as an excipient in the creation of precisely sized microparticles for injection to produce a sustained release profile by using short chain PLGA polymer allowing the PLGA to be manipulated during the formulation process without the use of organic solvents.

Other polymer excipients can be used if they are pharmaceutically acceptable and injectable. Another useful polymer is PDLLA which is poly-dl-lactic acid which has a higher glass transition point (about 45°-55° C.) than PLGA having a glass transition point of about 30-40° C.

Unlike previous approaches which has relied solely on the chemical composition of microparticles for injection as a means for creating controlled release formulations, the present invention relies additionally on precise sizing of the microparticles and the use of at least two different sizes of microparticles in the formulation. By exploiting the precise differences in surface area to volume ratio in the different populations of microparticles in the formulation, there is intrinsically less reliance on the chemistry of the particles to produce a sustained blood level of drug. By relaxing the requirement that the chemistry will have the predominant effect on the controlled release behavior a simpler chemistry can be employed which is easier and less costly to manufacture, and which avoids the use of organic solvents during its production period. For example, short chain PLGA polymer can be employed which can be processed without the use of organic solvents.

Poly (lactide-co-glycolides) (PLGA) compositions are commercially available from Boehringer Ingelheim (Germany) under the Resomer mark e.g. PLGA 50:50 (Resomer RG-502), PLGA 75:25 (Resomer RG-752) and d, t-PLA (Resomer RG-206) and from Birmingham Polymers (Birmingham, Ala.). These copolymers are available in a wide range of molecular weights and ratios of lactic acid to glycolic acid.

An aspect of the invention is that it is possible to create a formulation of spherical particles which formulation provides a desired drug release profile by combining a plurality of different groups of particles wherein each group consists of particles all of which have a known size, number and shape so that the combined groups of the formulation provide a rate of dissolution in a known environment to which the formulation will be delivered.

Another aspect of the formulation of the invention is that it be comprised of a plurality (2 or more) of different groups of particles wherein the particles within each group are substantially the same in size and shape (±110%) and are different from one group to another group as regards the drug release profile of the particles in a particular group. The particles preferably have a size in a range of from about 1 to about 100 micrometers in diameter and more preferably about 2 to 70 or 2 to 40 or 4 to 30 micrometers in diameter.

An advantage of the invention is that it is particularly suitable for the administration of drugs with a narrow therapeutic window so that the drug is released at a rate sufficient to provide a therapeutic result and insufficient to obtain a toxic effect.

Another aspect of the invention is that the particles within any given group are all of substantially the same size and coating thickness (±10%) and are spherical in shape.

Another aspect of the invention is that with overall volume limitations the duration of the release rate at a given level can only be extended by the use of coatings of different thicknesses or different dissolution rates.

It is an object of this invention to describe how encapsulated microspheres of fentanyl can be incorporated into an injectable formulation to produce three days of steady state fentanyl blood levels similar to those seen following application of the Duragesic fentanyl patch.

It is another object of this invention to show that by combining microspheres of different sizes into a single dosage form, the controlled sustained release rate of fentanyl can be achieved beyond three days and extending to as long as one month.

It is another object of this invention to show that, by incorporating encapsulated fentanyl microspheres of different sizes into the injectable formulation that overlapping defferential controlled release profiles can be exploited to more precisely produce a desired aggregate controlled release profile for fentanyl in the blood.

It is another object of this invention to show that the production of single use vials of the programmable controlled release fentanyl formulation being described, composed of lyophilized powder for reconstitution or of a gel for reconstitution, is a simple, low cost process.

It is another object of this invention to show that, by using an injectable slow release formulation of fentanyl, the entire prescribed dose is introduced into the patient's body without providing a mechanism for diversion after the dose has been given.

It is another object of this invention to show that the invention can be applied to the potent opioid analgesic drugs fentanyl and sufentanil.

It is another object of this invention to show that, when given as an intramuscular or subcutaneous injection, sufentanil microspheres, because sufentanil is ten times more potent than fentanyl, can treat chronic pain patients who are refractory to morphine and fentanyl therapy.

It is another object of this invention to show that it is advantageous to dose patients with an injection every three days rather than use patch therapy because diversion, compliance and cost issues are mitigated.

Another aspect of the invention is a method of reducing the diversion of narcotics by not using self-administered narcotics but rather by having a patient's caregiver (e.g. doctor or nurse) administer an injection to the patient while the patient is at the place of business of the caregiver (e.g. the doctor's office, clinic or hospital). The administered narcotic will be a controlled release formulation including but not limited to formulations as described here. More preferred formulations are injectable and provide for controlled release at therapeutic levels over 3 or more days, more preferable 7 or more days and still more preferably 2 weeks or more.

A method of providing pain management to patient in need of pain control while reducing the probability of diversion of narcotics is disclosed. The drug used may be any drug or combination of drugs intended for administration to patients (which are generally mammals and preferably humans) for controlling pain. The drug is formulated into microcapsules which are preferably provided in dry, unit dose form. Sterile containers of liquid are provided, preferably in containers allowing for sterile withdrawal of the liquid by a syringe for mixing with the microcapsules to create a dispersion. The dispersion is injected into a patient by the patient's caregiver. Once injected the microcapsules dissolve gradually and thereby gradually release drug to the patient. The microcapsules are designed so that drug will be released at a rate sufficient to provide for adequate pain control for the patient over a relatively long period of time, e.g. three or more days, preferably seven or more days, and more preferably fourteen or more days. Because the all of the drug provided to the patient is injected into the patient diversion of drug is virtually eliminated.

An aspect of the invention is that the patient's caregiver sees the patient frequently making is possible to closely monitor the patient to determine the patient's condition and the level of pain control be achieved.

Another aspect of the invention is that the patient is provided with pain management therapy over a relatively long period of time on an outpatient basis without providing the patient with drug that can be used by anyone other than the patient.

These and other features of the invention will become apparent to those persons skilled in the art upon reading the details of the invention as more fully described below.

### BRIEF DESCRIPTIONS OF THE DRAWINGS

FIG. 1 is a schematic view of a spray drying device.

FIG. 2 is a schematic view of an embodiment of an extrusion device used to create spherical particles.

FIG. 3 is a schematic view of an embodiment of an extrusion device used to create spherical coated particles.

FIG. 4 is a graph of time versus (amount of a compound dissolved minus the amount eliminated) for a single particle or group to substantially identical particles.

FIG. 5 is a graph of time versus (amount of a compound dissolved minus the amount eliminated) for two different particles or two different groups of particles where the particles within a given group are substantially identical and also showing the combined effect of the two groups.

FIG. 6 is a graph of time versus (amount of a compound dissolved minus the amount eliminated) for three different particles or three different groups of particles where the particles within a given group are substantially identical and also showing the combined effect of the three groups.

### DETAILED DESCRIPTION OF THE PREFERRED EMBODIMENTS

Before the present formulations and methods are described, it is to be understood that this invention is not limited to the particular components and steps described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “and,” and “the” include plural referents unless the context clearly dictates otherwise. Thus, for example, reference to “a particle” includes a plurality of particles and reference to “a fluid” includes reference to a mixture of fluids, and equivalents thereof known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Invention in General—Formulations

The formulations and methods for creating formulations of the invention are based in mathematics. For any given particle having a given amount of surface area the rate of dissolution will decrease as the particle dissolves and the total available surface area decreases. Thus, a spherical particle with two square units of available surface area which dissolves at a rate of X per unit of time will be dissolving at a rate of X/2 per unit of time once the particle has dissolved so that it has one square unit of available surface area. This assumes a constant environment unaffected by the dissolution.

By combining two different particles each comprised of the same material but of a different size the combined rate of the two particles together is different from either particle by itself. The combined rate of a small and a large particle is slower than two large particles and faster than two small particles.

A particle with a large available surface area has a more rapid dissolution rate that a particle with a small available surface area. However, assuming the same total volume in two groups of particles the group of smaller particles has a faster dissolution rate than the group of larger particles because the group of smaller particles will have a larger available surface area than the group of larger particles.

It is often desirable to deliver a predetermined amount of compound (such as a drug) to a system (such as a human) at a rate which maintains the compound in the system at a desired level over a desired period of time. When the total amount (weight and volume) is fixed the rate of dissolution is dictated by the available surface area. One spherical particle with a given total volume will present approximately half the surface area as ten particles with the same combined volume as the one particle. Each time the number of particles is increased by a multiple of ten (and the combined volume remains constant) the total available surface area approximately doubles. The following provides specific examples of how the total available surface area increases as the same total volume (e.g. a drug) is included in larger numbers of spheres.

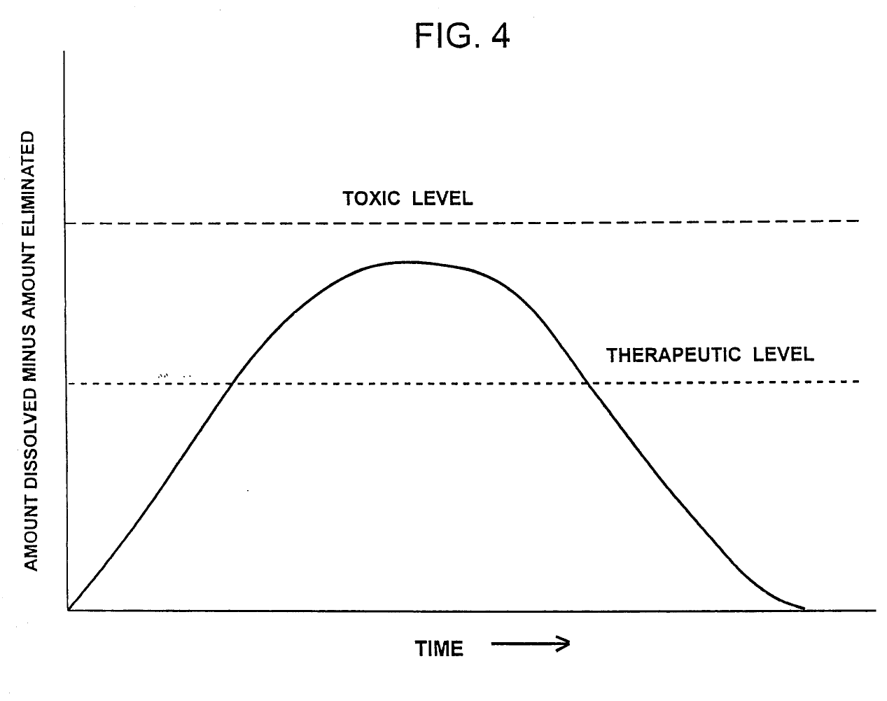

Formulations of the invention may include some narcotics (e.g. fentanyl) for immediate release to provide quick pain relief to the patient. Further, greater numbers of groups of different particles can increase the duration time drug is released and decrease changes in blood levels over time. Thus, 2 or more, 3 or more, 4 or more or 5 or more groups can be used to maintain the desired therapeutic level over time—see FIGS. 4, 5 and 6.

### Invention in General—Reducing Narcotic Diversion

A method of doing business whereby diversion of narcotics is reduced is provided for here. The narcotics are stored in a dry form, in a secure location, under strict inventory control. The caregiver only, and not the patient nor others generally in contact with the patient have access to the narcotic. The caregiver mixes the dry powder with a liquid as described here to create a suspension formulation. The formulation is injected into the patient and provides a desired level of narcotic to the patient over a desired period of time, e.g. 3 to 7 days or 1 to 4 weeks. The patient is monitored and re-visits the caregiver for injections over time as needed.

### Specifics of Particle Sizes

It is convenient to provide an oral pharmaceutical dose in a single unit, e.g. pill or capsule. The total volume of the capsule is limited by the amount a patient can easily swallow. For humans the upper limit is about 2 cubic centimeters. Assuming a particular oral dosage form will contain a total volume of 2 cubic centimeters the size a single sphere which will hold a 2 cc volume can be readily calculated using the formula for the volume of a sphere as follows:

Volume of a sphere=(4/3)π r³ if the volume of a sphere is 2 cc then

2 cc=(4/3)π r³

2=(4/3) 3.14159 r³

2=4.1887867 r³

0.477645=r³

0.781592 cm=r

r=7,815 micrometers

diameter=d=2r=15,630 micrometers

The formula for the surface area of a sphere is 4π r². Because “r” was found to be 0.781592 cm the surface area =4(3.14159)(0.781592)=9.8217 cm²

The formula for the volume of a sphere can be readily modified to determine the volume of any number of spheres “n” needed to make a total volume of 2 cubic centimeters.

2 cc=n(4/3)π r³

This formula was solved above for “n” equals “1” and can be solved for any “n.” For example, when “n” is 10 the formula becomes

2 cc=10(4/3)π r³

2 cc=10(4/3) 3.14159 r³

2 cc=41.887867 r³

0.0477645=r³

0.362783 cm=r³

r=3627 micrometers

d=7254 micrometers

The volume of each sphere is 0.2 cm³ and the surface area of each sphere is 1.65388 cm². Thus, the total volume of the 10 spheres remains the same (i.e. 2 cc) but the surface area of all 10 spheres is 16.5 cm² as compared to 9.8217 cm² when “n” was one.

When “n” equals 100 the radius “r” can be solved for and found to be 0.1684 cm with the volume of each of the 100 spheres being 0.02 cm³. The surface area of each sphere is 0.3563 cm² and the combined surface area of all 100 spheres is 35.63 cm²—the combined volume remains the same at 2 cm³. The equations for the surface area and volume can be used to solve for the radius “r” and diameter “d” of any number of spheres “n” which equal a total volume of 2 cm³ and the results are provided below.

TABLE 1
Total volume is 2 cm³

| | | | | |
|---|---|---|---|---|
| | | | | Surface Area |
| | r(micro- | | Surface area | Volume |
| n | meters) | D | (cm²) | (cm⁻¹) |

| | | | | |
|---|---|---|---|---|
| 1 | 7815 | 15,630 | 9.8217 | 4.91085 |
| 10 | 3627 | 7,254 | 16.5 | 8.25 |
| 100 | 1684 | 3,378 | 35.63 | 17.815 |
| 1,000 | 781 | 1,562 | 76.766 | 38.383 |
| 10,000 | 362 | 724 | 165 | 82.5 |
| 100,000 | 168 | 336 | 356 | 178 |
| 1,000,000 | 78 | 156 | 768 | 384 |
| 10,000,000 | 36 | 72 | 1,653 | 826.5 |
| 100,000,000 | 16.8 | 33.6 | 3,563 | 1781.5 |
| 1,000,000,000 | 7.8 | 15.6 | 7,677 | 3838.5 |
| 10,000,000,000 | 3.6 | 7.2 | 16,539 | 8269.5 |
| 100,000,000,000 | 1.6 | 3.2 | 35,631 | 17815.5 |

From the above it can be seen that when “n” is increased by a factor of 10 and total combined volume is maintained constant at 2.0 and the combined surface area of all of the spheres increases by approximately a factor of 2 for each increase of 10× for n.

Although the surface area approximately doubles as “n” increases by a factor of ten the absolute effect of the doubling is small when “n” is increased from 1 to 10 to 100. Specifically, the increase in surface area from 9.8 to 16.5 is only an increase of 6.7 cm² and from 16.5 to 35.6 is only an increase of 19.1 cm². However, when “n” increases from 10⁹ to 10¹⁰ the surface area increases from 7677 to 16,539 resulting in an increase of 8,862 cm². When “n” increases from 10¹⁰ to 10¹¹ the surface area increases from 16,539 to 35,631 resulting in an increase of 18,992 cm².

For “n” at the extremes of the calculations provided above the gross increase in surface area is as follows:

TABLE 2

| N | gross increase in surface area (cm²) |
|---|---|
| 1 to 10 | 6.7 |
| 10 to 100 | 19.1 |
| 10⁹ to 10¹⁰ | 8,8863 |
| 10¹⁰ to 10¹¹ | 18,992 |

The larger the available surface area the faster the rate of dissolution of the solute rug assuming the solvent is not saturated. In nearly all situations the solute drug will only be administered to the surrounding environment of the solvent (e.g. blood or G.I. tract) in relatively small amounts. Accordingly, the solvent never approaches saturation.

Formulations of the invention are described and claimed here and such formulations may have two, three or a plurality of different groups of particles therein. The formulation suspension may be created where a first group has a first surface area and a second group has 1,000 square centimeters or more surface area than the first group or e.g. 2,000 or more; 5,000 or more; or 10,000 or more square centimeters of surface area more than the surface area of the first group. Formulations of suspensions of particles may be created whereby a plurality of different groups are present and the total surface area of any one group different from the total surface area of any other group by a desired amount e.g. 1,000; 2,000; 3,000; 4,000; 5,000; and 10,000 or more square centimeters of surface area.

Using data such as generated in Table 1 and the results of Table 2 a formulation of the invention can be created which provides a desired release profile. The solvent is the surrounding environment which can be any area where the drug is delivered including the blood, the G.I. tract, the surface of the lungs, mucosal surfaces on the gums, as well as in nasal, anal and vaginal areas. The solvent or surrounding environment into which the drug is administered can be assumed to be known within a given environment (e.g. blood) in a given species of animal (e.g. human). Thus, the unknown that remains is the rate of dissolution of a particle of known size in a given solvent. After calculating the rate of release “R” (weight or volume dissolved per unit of time) for a known particle size the rate of dissolution of other particle sizes with different available surface areas can be calculated. Assuming all the particles of a group of particles are spherical and also assuming that the particles in a given group of particles all have substantially the same size (available surface area), the rate of dissolution of a group of particles can be readily determined. Using this information a formulation can be created with different groups or types of particles wherein each group of particles has a known drug release profile within the environment the formulation is delivered to. The formulation preferably comprises a number of different groups which release drug at different rates and/or times and provide a desired drug release profile, e.g. substantially constant blood levels over a therapeutically effective time period.

Calculations are provided below in Tables 3, 4 and 5 respectively for total volumes of 1 cm³, 0.5 cm³ and 0.1 cm³ which are volume sizes that might be used for typical dosages of orally administered pharmaceutically active compounds.

TABLE 2
Total volume is 1 cm³

| | radius | diameter | | Surface area |
|---|---|---|---|---|
| number of | (micro- | (micro- | Surface area | Volume |
| spheres | meters) | meters) | (cm²) | (cm⁻¹) |
| 1 | 6203.5 | 12407.0 | 4.84 | 4.8 |
| 10 | 2879.4 | 5758.8 | 10.42 | 10.4 |
| 100 | 1336.5 | 2673.0 | 22.45 | 22.4 |
| 1,000 | 620.4 | 1240.7 | 48.36 | 48.4 |
| 10,000 | 287.9 | 575.9 | 104.19 | 104.2 |
| 100,000 | 133.7 | 267.3 | 224.47 | 224.5 |
| 1,000,000 | 62.0 | 124.1 | 483.60 | 483.6 |
| 10,000,000 | 28.8 | 57.6 | 1041.88 | 1041.9 |
| 100,000,000 | 13.4 | 26.7 | 2244.66 | 2244.7 |
| 1,000,000,000 | 6.2 | 12.4 | 4835.98 | 4836.0 |
| 10,000,000,000 | 2.9 | 5.8 | 10418.79 | 10418.8 |
| 100,000,000,000 | 1.3 | 2.7 | 22446.61 | 22446.6 |

TABLE 4
Total volume is 0.5 cm³

| | radius | diameter | | |
|---|---|---|---|---|
| number of | (micro- | (micro- | Surface area | Surface area |
| spheres | meters) | meters) | (cm2) | Volume |
| 1 | 4923.7 | 9847.5 | 3.05 | 6.1 |
| 10 | 2285.4 | 4570.8 | 6.56 | 13.1 |
| 100 | 1060.8 | 2121.6 | 14.14 | 28.3 |
| 1,000 | 492.4 | 984.7 | 30.46 | 60.9 |
| 10,000 | 228.5 | 457.1 | 65.63 | 131.3 |
| 100,000 | 106.1 | 212.2 | 141.40 | 282.8 |
| 1,000,000 | 49.2 | 98.5 | 304.65 | 609.3 |
| 10,000,000 | 22.9 | 45.7 | 656.34 | 1312.7 |
| 100,000,000 | 10.6 | 21.2 | 1414.05 | 2828.1 |
| 1,000,000,000 | 4.9 | 9.8 | 3046.47 | 6092.9 |
| 10,000,000,000 | 2.3 | 4.6 | 6563.43 | 13126.9 |
| 100,000,000,000 | 1.1 | 2.1 | 14140.48 | 28281.0 |

TABLE 5
Total volume is 0.1 cm³

| | radius | Diameter | | |
|---|---|---|---|---|
| number of | (micro- | (micro- | Surface area | Surface area |
| spheres | meters) | meters) | (cm2) | Volume |
| 1 | 2879.4 | 5758.8 | 1.04 | 10.4 |
| 10 | 1336.5 | 2673.0 | 2.24 | 22.4 |
| 100 | 620.4 | 1240.7 | 4.84 | 48.4 |
| 1,000 | 287.9 | 575.9 | 10.42 | 104.2 |
| 10,000 | 133.7 | 267.3 | 22.45 | 224.5 |
| 100,000 | 62.0 | 124.1 | 48.36 | 483.6 |
| 1,000,000 | 28.8 | 57.6 | 104.19 | 1041.9 |
| 10,000,000 | 13.4 | 26.7 | 224.47 | 2244.7 |
| 100,000,000 | 6.2 | 12.4 | 483.60 | 4836.0 |
| 1,000,000,000 | 2.9 | 5.8 | 1041.88 | 10418.8 |
| 10,000,000,000 | 1.3 | 2.7 | 2244.66 | 22446.6 |
| 100,000,000,000 | 0.6 | 1.2 | 4835.98 | 48359.8 |

### Particle Formation Methodlogy

Particles and coated particles can be produced via any available technology.

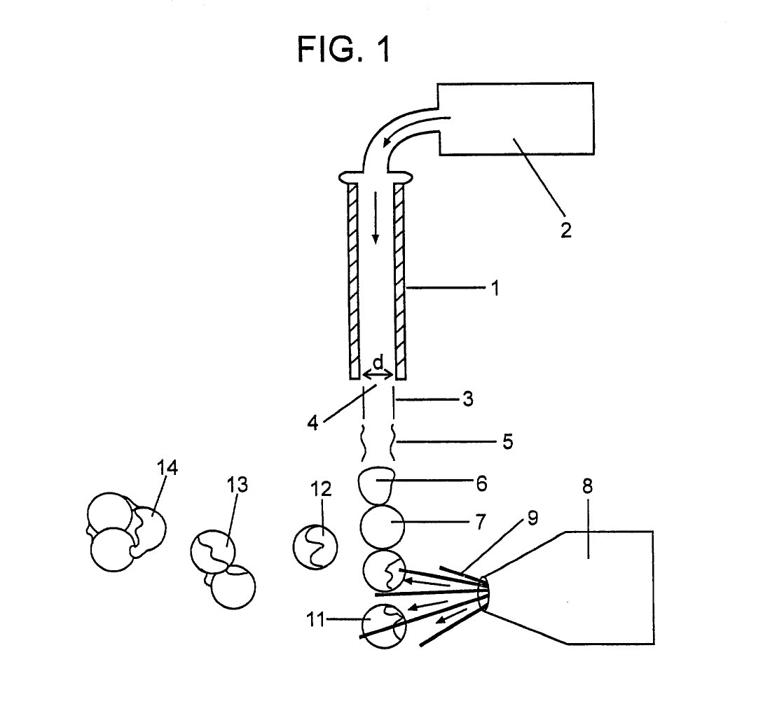

Referring to FIG. 1, cylindrical tube 1 is shown in fluid connection with a liquid source 2 which can supply liquid 3 to the tube 1. The liquid 3 exits the tube 1 from an exit opening which can be any configuration but is preferably circular and has a diameter D.

The liquid 3 exits the opening 4 and forms a stream which breaks into segments 5 and eventually forms partial spheres 6 and then spheres 7 which are substantially equal in size and shape. The spheres 7 could be used in creating a formulation of the invention. Different size spheres from different sized tubes 1 could create different groups of spheres as needed for a desired dissolution profile.

The processing of FIG. 1 can stop at the formation of the particles 7. However, in order to attempt to obtain a dissolution profile which achieves a longer steady state level of the desired compound a coating is often used. The coating source 8 creates a spray 9 of a coating material which is brought into contact with and sticks to particles 10, 11 and 12 often in different amounts. Further, two particles 13 may become coated together or three or more particles 14 may become coated together.

The result is a random mixture of particles coated to different degrees and combined with different numbers of other particles. Although coated particles of this type could be used to create controlled release formulations they are not particularly useful in creating formulations of the present invention because of the random nature of the resulting mixture of coated particles. The coating material can be mixed with rather than sprayed on the particles and a similar random mixture of coated particles and coated groups of particles will result. The random mixture has some advantages. It can provide a greater range of release rates than a single type of particle. The greater range of release rates may provide a release profile which is desirable. However, considerable trial and error is required in producing a desired release profile. Further, great care must be taken once the desired profile is obtained in repeating all preparation steps precisely from batch to batch. Otherwise, each new batch of formulation produced will have a different release profile.

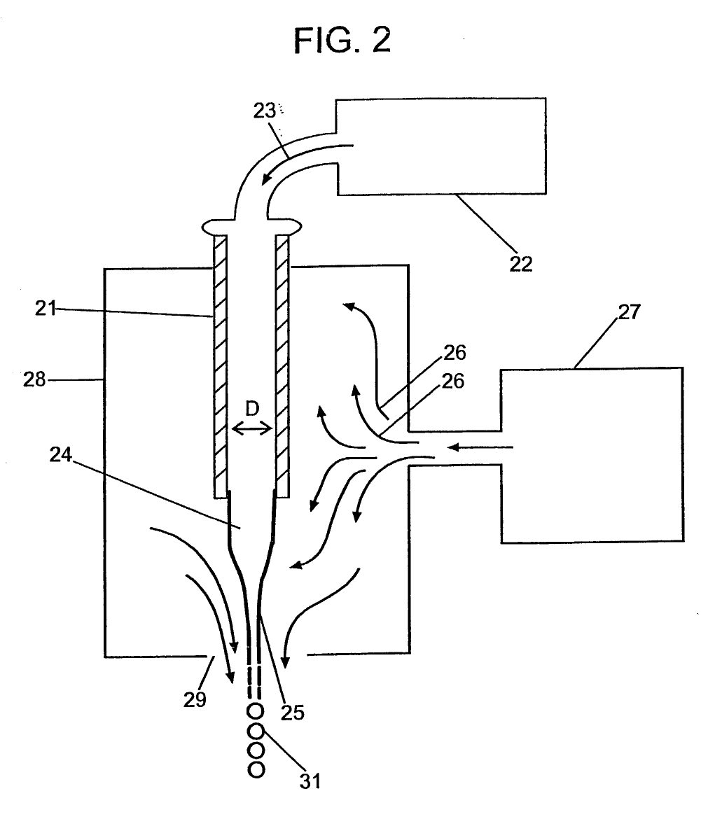

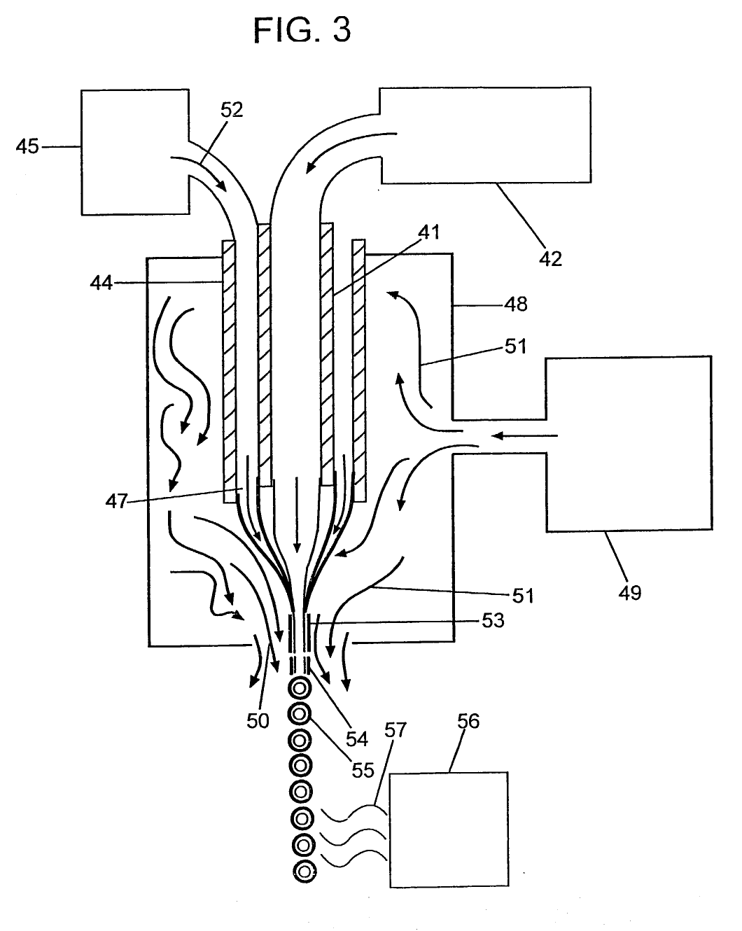

The process for producing particles 7 as shown in FIG. 1 has yet another disadvantage or limitation. Specifically, the diameter D of the tube 1 dictates that the diameter of the particles 7 formed will be approximately D×1.89 (Rayleigh, “On the instability of jets”, Proc. London Math. Soc., 4-13, 1878). Thus, when attempting to make very small particles (e.g. less than 20 micrometers) the inside diameter of the tube 1 must be very small. Not only is it difficult to manufacture tubes with such a small diameter but the narrower tubes tend to clog easily. These problems can be solved by using a different technology for producing particles and coated particles as shown in FIGS. 2 and 3.

FIG. 2 shows a tube 21 supplied by a liquid source 22. The liquid 23 flows out of the exit 24. The liquid 23 stream is focused to a narrowed stable jet 25 by a gas 26 provided by the gas source 27 flowing into a pressure chamber 28 and out of an exit orifice 29. The jet 25 disassociates into segments 30 which form spheres 31 in the same manner in which the stream of liquid 3 forms the spheres 7 shown in FIG. 1. However, the spheres 31 have a diameter which is 1.89×the diameter Dⱼ of the jet and not 1.89×the diameter D of the tube 21. The diameter of the jet 25 (Dⱼ) is substantially smaller than the diameter D of the tube 21. Thus, the system of FIG. 2 can be used to make very small particles as compared to the system of FIG. 1 without clogging the exit 24 of the tube 21 because the diameter D of the tube 21 can remain large—and without clogging the exit orifice 29 of the pressure chamber 28 because the jet 25 exits the orifice 29 surrounded by the gas 26.

The particles 31 can be coated using a spray on coating as shown in FIG. 1. However, similar problems occur as described above with reference to FIG. 1. The particles 31 can be used without any coating. Groups of particles can be combined to provide a desired dissolution profile. The small size of the particles provides certain advantages as shown in Tables 1-5. Particles in a size range of 1-20 micrometers can not be easily produced in a system as shown in FIG. 1 and particles in this size range provide the greatest differences in surface areas—see Tables 1-5 and Table 2 in particular. However, the particles themselves (without a coating) are limited in terms of the dissolution profile they can produce particularly when the total volume of the particles in a formulation is limited. Thus, a coating is preferred and a preferred means of obtaining such is shown in FIG. 3.

The system schematically shown in FIG. 3 includes a tube 41 in fluid connection with a liquid source 42 which supplies liquid 43 to the cylindrical channel of the tube 41. A tube 44 is concentrically positioned around the tube 41 and is in fluid connection with a coating source 45. The exit opening 46 of the tube 41 and the exit opening 47 of the tube 44 are both positioned inside of a pressure chamber 48. The chamber 48 is in fluid connection with the gas source 49 which flows out of the exit orifice 50 of the chamber 48. The gas 51 focuses the streams of liquid 43 and coating 52 into a stable jet 53. The jet 53 disassociates into segmented streams 54 of liquid 43 concentrically surrounded by coating 52. The segmented streams 53 form spheres 55.

The spheres 55 are comprised of a liquid 43 center surrounded by a coating 52. The spheres 55 are preferably very small, e.g. a diameter of less than 50 μm, preferably less than 20 μm and more preferably about 10 μm. The smaller the particles the more readily evaporation will take place which will cure or solidify the coating 52.

An energy source 56 may be used to direct energy 57 onto the particles 55 to enhance the rate of curing, hardening, evaporation, etc. The energy 57 may be any type of energy including heat, forced air, I.R. or U.V. light etc. alone or in combination. Some polymer materials are designed to be cured using a particular frequency of light. The light can be directed, focused and/or intensified using lenses, mirrors and the like to obtain a desired result.

The coated particles 55 can include any liquid 43 coated with any coating material 52. However, in accordance with the present invention it is preferable that the liquid 43 be comprised of a pharmaceutically active drug which is most preferably an opioid drug and still most preferably fentanyl or sufentanil. Further, the coating material can be comprised of any type of material which can be cured, dried or fixed in any fashion in order to form an outer spherical coating around the center. However, it is preferable that the coating material be comprised of a polymer material and more preferable if the polymer material is quickly and readily curable and is a material which is commonly accepted as useful as a carried material in controlled release formulations used in pharmaceutical applications. A number of such polymer materials are disclosed within the Background of the Invention section and publications cited there.

To form a coated particle 55 the liquid 43 is forced through the channel of the tube 41. The liquid is preferably a relatively high concentration of a drug such as fentanyl in either an aqueous or alcohol based solvent or other solvent which will quickly evaporate (e.g. ether). The exit opening 46 of the tube 41 and the exit opening 47 of the tube 44 are both positioned inside the pressure chamber 48. The coating material 52 is initially in a liquid form and is forced through the exit opening 46 of the tube 44 which is positioned concentrically around the tube 41 in a manner which causes a stream of the liquid coating material to be expelled from the opening 47 at substantially the same velocity as the liquid 43 is forced from the opening 46 of the tube 41. Accordingly, the stream of the coating material is concentrically positioned around the stream of the center liquid 43. The streams exit the openings of the two concentrically positioned tubes as a single combined stream which then disassociates into segments streams 53 which segments form the cooled spheres 55.

In order for the spheres to be made small it is necessary to use the gas from the gas source 49 forced into the pressure chamber 48 in a manner which causes the gas to exit the pressure chamber 48 downstream of the concentrically positioned streams exiting the tubes 41 and 44. It is preferable for the density of the liquid 43 to be substantially the same as the liquid of the coating 52. This allows the gas from the gas source 49 to focus the concentrically positioned streams into a stable unified jet which flows out of the chamber 48 breaking up into segments and thereafter forming the spherical coated particles 55 of the coating material surrounding the center of pharmaceutically active drug.

In accordance with the invention the gas from the gas source forms the stable jet and the diameter of the jet is substantially smaller than would be the case if the gas were not focusing the streams exiting the tubes 41 and 44. The diameter of the jet is defined by the following formula:

$${d}_{j} ≅ {( \frac{8 {ρ}_{1}}{{π}^{2} Δ {P}_{g}} )}^{1 / 4} {Q}^{1 / 2}$$

wherein dⱼ is the diameter of the stable unified jet, indicates approximately equally to where an acceptable margin of error is ±10%, ρ₁ is the average density of the liquid of the jet and ΔP_{g} is change in gas pressure of gas surrounding the stream at a given point A at the exit and Q is the total flow rate of the stable unified jet.

By using the technology described above and shown in FIGS. 2 and 3 it is possible to form very small and very uniform particles. The particles may be of any size but are preferably in less than 100 micrometers in diameter, more preferably less than 50 micrometers in diameter and still more preferably less than 20 micrometers in diameter. The technology described above and shown in FIGS. 2 and 3 is capable of producing particles which are as small as approximately 1 micrometer in diameter and preferred formulations of the invention will include particles which have a diameter of approximately 10 micrometers. The sphere forming technology can produce particles which are substantially identical in shape (spherical) and substantially identical in size ±10% variation in the particle diameter, more preferably ±3% and still more preferably ±1% variation in particle diameter where the particle may have a diameter as small as 1 μm or more or as large as 100 μm or more.

Those skilled in the art will understand that in addition to the tubes 41 and 44 a plurality of additional concentrically positioned tubes may be added to the system. This would make it possible to add additional coating materials or include additional active components surrounded by outer shells of coating material. Those skilled in the art will understand that the system works best when the Weber Number is in a range of from about 1 to about 40 wherein the Weber Number is defined by the following equation:

$$We = \frac{{ρ}_{g} {V}_{g}^{2} d}{γ}$$

wherein the ρ_{g} is the density of the gas, d is the diameter of the stable microjet, γ the liquid-gas surface tension and V_{g}² is the velocity of the gas squared. More preferably the Weber number is in a range of about 5 to about 25.

Further, those skilled in the art will understand that it is preferable for the Ohnesorge number to be less than 1, wherein the Ohnesorge number (Oh) is defined by

$$Oh = \frac{{μ}_{1}}{{( {ρ}_{1} γ d )}^{1 / 2}}$$

wherein μ₁ is the velocity of the liquid, ρ₁ is the density of the liquid and d is the diameter of the stable capillary microjet.

Those skilled in the art will also understand that the method for producing particles and coated particles as described above is best carried out when the difference in the pressure between the pressure chamber exit orifice is equal to or less than 20 times the surface tension of the liquid comprising the coating material with the gas, divided by the radius of the stable unified jet. Details relating to the technology are described within issued U.S. Pat. No. 6,234,402 issued May 22, 2001 and incorporated herein by reference. Those skilled in the art will understand that some adjustments may be made in the density and velocity of the different fluids and gases used in order to obtain the desired result in terms of the fluid—fluid interfaces including the particle interface between the coating material and the inner liquid material as well as the stable interface between the gas and the coating material. It is desirable to obtain the stable microjet stream which has substantially no aberrations or pertubations in the stream making it possible for the stream to disassociate into very uniform size and shaped particles. This systems shown in FIGS. 2 and 3 make it possible to maintain a stable liquid-gas interface between the outer surface of the liquid or coating material and the gas thereby forming a stable jet which is focused on the exit orifice of the pressure chamber resulting in particles which have very small deviation in terms of diameter from one particle to the next. It is also possible to create hollow particles and to reverse the positioning of the different fluids. For examples, the center tube can be used to supply gas whereas the pressure chamber can be used to supply a liquid. The technology for such is described within issued U.S. Pat. No. 6,196,525 issued Mar. 6, 2001 which patent along with other patents cited herein is incorporated in its entirety.

### Dissolution Profiles

When any particle dissolves in any solvent the amount of solute in the solution increases over time. However, some solvents are present in systems where the portion of the dissolving solute is being removed from the solution. This could take place in a chemical reaction where a portion of the dissolved solute reacts with another components present in the system. However, the most typical situation is where a drug present in blood is metabolized which subtracts solute drug from the solvent blood. In any such system the dissolution profile over time shows an increase followed by a steady state followed by a decrease as is shown by the solid line in FIG. 4. It is desirable to maintain the level of a drug above the therapeutic level shown by the line of short dashes but below the toxin level shown by the line of long dashes. Maintaining the level of drug in a desired range for a significant period is difficult to obtain particularly when using a single type of particle.

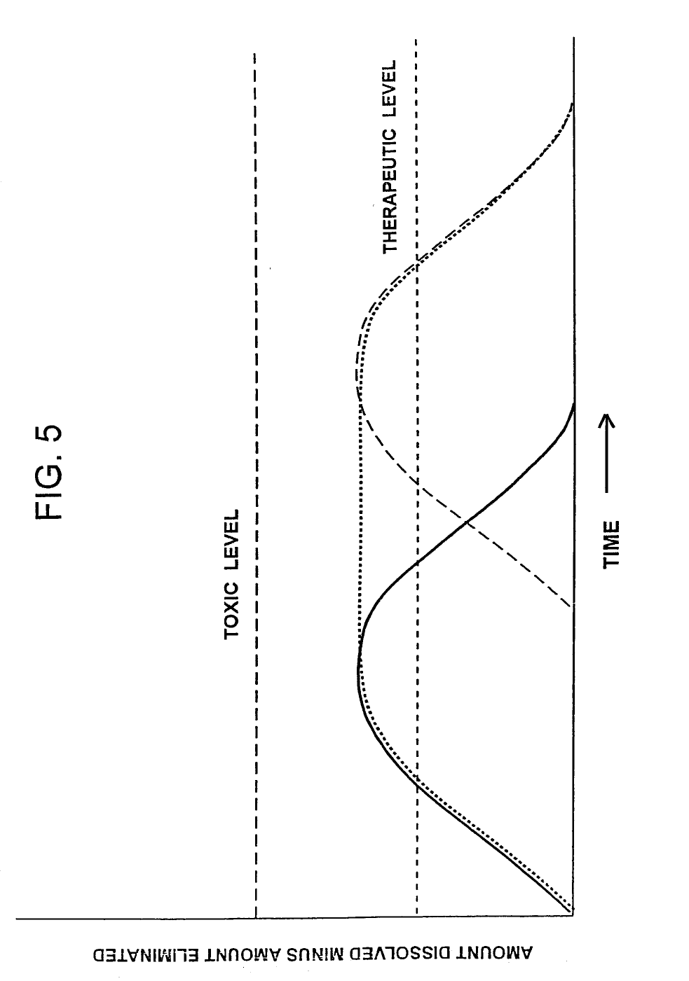

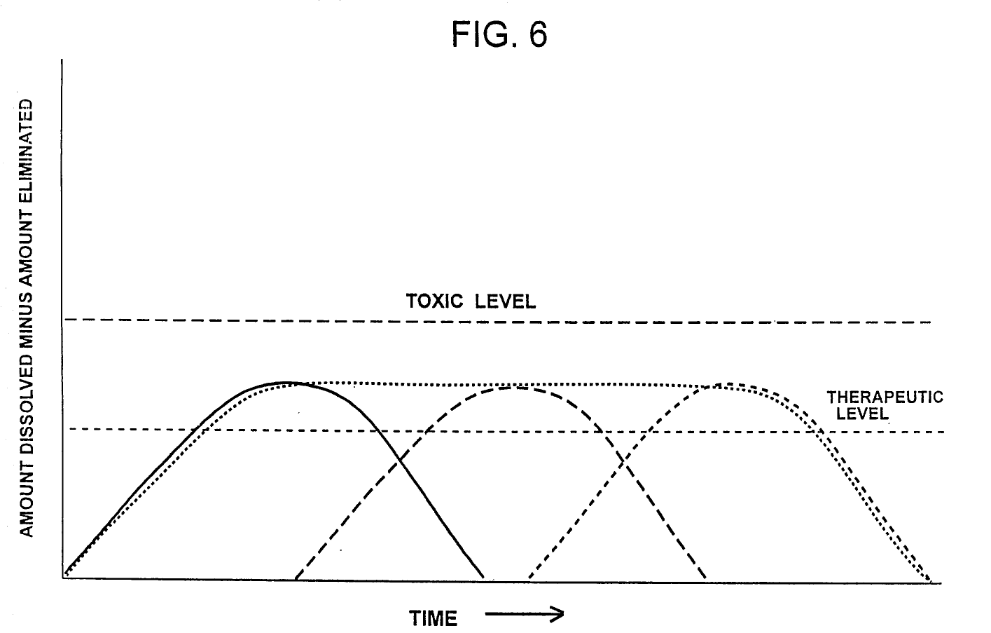

FIG. 5 shows how the therapeutic level can be maintained over a longer period of time using two different types of particles. In FIG. 5 the independent effect of a first type of particle is shown by the solid line. The dashed curve shows the independent effect of a second type of coated particle. The dotted curve shows the combined effect of the two types of particles. When the particle of the first type are completely dissolved and are being metabolized out of the system the coatings on the particle of the second type have dissolved and the rate of dissolution matches the rate at which all drug in the system is being metabolized out of the system. Thus, a longer steady state period is maintained. This effect is further enhanced using three different types of particles as shown in FIG. 6.

An effect similar to that shown in FIGS. 5 and 6 might be obtained by dosing the patient with a single type of particle but with two or three doses administered at different times. However, the dosing intervals would need to be precisely timed and patient compliance would be difficult to obtain. Further, by decreasing the number of times a drug is administered the expense and discomfort to the patient is reduced particularly when the formulation is administered by injection.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

There are corporations with specific expertise in drug delivery technologies including controlled release oral formulations such as Alza corporation and Elan. A search of patents, published patent applications and related publications will provide those skilled in the art reading this disclosure with significant possible controlled release oral formulations. Examples include the formulations disclosed in any of the U.S. Pat. Nos. 5,637,320 issued Jun. 10, 1997; 5,505,962 issued Apr. 9, 1996; 5,641,745 issued Jun. 24, 1997; and 5,641,515 issued Jun. 24, 1997. Although specific formulations are disclosed here and in these patents the invention is more general than any specific formulation. This includes the discovery that by placing pharmaceutically active drug in a controlled release formulation which maintains therapeutic levels over substantially longer periods of time as compared to quick release formulations, improved unexpected results are obtained.

### Particles Formed Using Supercritical Fluid Precipitation

The devices, systems and methodology disclosed and described above in connection with FIGS. 2 and 3 can also be used in combination with supercritical fluid precipitation technology of the type described within U.S. Pat. Nos. 6,063,910 issued May 16, 2000; 5,766,637 issued Jun. 16, 1998; 6,228,394 issued May 8, 2001; and 6,095,134 issued Aug. 1, 2000 all of which are incorporated herein by reference in their entirety. Basically, the technology utilizes a supercritical fluid such as liquid CO₂ in order to form solid particles of a material such as a drug or a protein for use in a formulation.

Referring to FIG. 2 the gas source 27 could be replaced with a liquid CO₂ and the liquid CO₂ could become the focusing fluid. The liquid 23 supplied into the tube 21 could be any liquid comprised of any desired material. However, the liquid 23 would preferably be a liquid which included an active compound such as a drug which is dissolved within a solvent such as water and further combined with a solvent such as ethanol. The solvent liquid 23 is focused by the surrounding liquid 26 which may be CO₂. When the CO₂ exits the pressure chamber 28 via the orifice opening 29 the rapid evaporation draws the liquid water and ethanol away leaving dry particles 31.

Referring to FIG. 3 it would also be possible to use supercritical fluids in place of the coating 52 or in place of the gas 51. Those skilled in the art will recognize that a variety of different combinations of liquids, gases, solutions and supercritical fluids are possible using the systems as shown and described above with respect to FIGS. 2 and 3 particularly when taken in combination with the above-referenced patents which disclose basic technology used in the field of supercritical field precipitation.

### Fentanyl

Steady state blood levels of fentanyl have proved instrumental in the management chronic cancer pain. Because fentanyl is 100 times more potent than morphine, is has oven invaluable for treating pain in patients requiring large quantities of oral morphine each day for pain control. Typically, these patients have a cancer diagnosis and see their morphine requirements increase over time. Potent drugs more efficiently interact with target receptors and therefore can more effectively treat pain in patients who have developed a tolerance for morphine.

Because fentanyl cannot be absorbed via the GI tract, chronic steady state blood levels are achieved through continuous intravenous infusion. Transdermal delivery of fentanyl has proven to be a safe and effective way to establish and maintain therapeutic blood levels of fentanyl in patients with severe chronic pain requiring large amounts of daily opioid analgesics in order to obtain pain relief. The level for pain control may be 1 microgram per ml of blood ±20%.

The Duragesic® Fentanyl TTS transdermal patch is specifically designed to produce therapeutic blood levels of the fentanyl, an opioid analgesic 100 times more tent than morphine. The Duragesic patch is sold in four different dosage strengths: 25 μg/hour, 50 μg/hour, 75 g/hour and 100 μg/hour. A liquid reservoir integrated into the patch contains a three day supply of fentanyl. Because the transfer of fentanyl across the in is not completely efficient, more than (three days)×(24 hours/day)×(μμg/hour) of total packaged fentanyl is required. For example, the Duragesic 100 μg/hour product contains 10 mg of fentanyl, or 100 hourly doses of 100 μg each even though only 3×24=72 hourly doses or 7.2 mg would be required assuming 100% transfer efficiency from the patch through the skin over labeled dosing period of three days.

Although the Duragesic patch has been well received by physicians and patients, the product suffers from problems which have limited its widespread use. Principal among those problems are: cost and issues associated with potential misuse and abuse of the large amount of potent narcotic stored within the Duragesic patch.

Duragesic fentanyl has been ranked as the most expensive opioid analgesic on the market today. Three studies published in the United States each place Duragesic as the most costly choice for pain management when dosage costs of analgesics are compared.

Fentanyl drug dosage forms produced using the present invention could be programmed to produce release profile similar to that of the Duragesic patch. Injectable controlled release formulation producing 25 μg/hour, 50 μg/hour, 75 μg/hour, 100 μg/hour or more could be created by combining microspheres of different sizes in the proper proportions into the dosage form. For example, for a seven day release profile, and assuming a 60% overall delivery efficiency, 26.88 mg of fentanyl could be incorporated to the dosage form for continuous release over a seven day period.

As with the Duragesic patch, fentanyl base is the preferred dosage form for use in the present invention. This is because fentanyl base is non-polar and more readily combined with excipients such as PLGA.

Sufentanil is a potent synthetic narcotic approximately ten times more potent than fentanyl. Its increased potency makes sufentanil more efficacious even at equipotent doses for patients who are resistant to narcotic therapy.

A seven day release sufentanil dosage form designed to release 10 μg/hour would be clinically equivalent to a fentanyl dosage form of 100 μg/hour in narcotic naive patients.

Sufentanil formulations with the present invention can be created in the same way as fentanyl formulations. A key difference being the requirement for 10 times less active drug in the formulation. For a seven day release profile of 10 μg of sufentanil per hour, 27 mg of sufentanil would be required in the drug dosage form.

Duragesic is intended for self application and is supplied in boxes of five patches each, patients must be compliant and not inadvertently apply more than one patch at a time. Again, the large amount of fentanyl in each patch form could quickly add up to a lethal dose if multiple patches were inappropriately applied.

Although it would be desirable to deliver more potent opioid analgesics such as sufentanil via transdermal patch, more precision delivery is required for these more potent compounds. Motivating the need for more potent pain drugs is the increasing number of patients who become progressively more refractory to opioid therapy and who can only be effectively treated by very potent opioid drugs.

The present invention also decreases diversion of narcotics by having the controlled release injectable formulation injected into the patient by the caregiver in the caregiver's place of business.

Fentanyl, a weak base, is sold for injection as the citrate salt, and is a polar molecule in solution at neutral pH. Because the Duragesic patch relies on driving fentanyl through the skin across a concentration gradient and because the skin is a non-polar, lipophillic material, the Duragesic patch contains fentanyl in its non-polar, free base form. Regardless of how the fentanyl is packaged or delivered, it is active in the blood as the free cation.

Fentanyl citrate is sold for intravenous, subcutaneous or intramuscular injection. When given intravenously, the half life is shorter than if the drug is effective “depoized” by injection intro the muscle or subcutaneous space. The pharmacokinetic profile (PK profile) is defined as the blood concentration over time and can be modified by combining a drug with an excipient designed to slow release from the injection site. Insulin, for example, was formulated into a zinc protamine preparation to slow its release from the subcutaneous space which has allowed people with diabetes to achieve therapeutic insulin blood levels while they sleep by taking a single long acting injection before bed time. The fentanyl used in formulations of the invention may be in any form as pharmaceutically acceptable salts, e.g. citrate salt, acetate salt.

Excipient selection has been the primary way that slow-release formulations of injectable drugs have been developed. Encapsulated particles of the invention use a method which modifies the geometric relationship between the active drug and excipient by precisely encapsulating the active drug with excipient into microspheres of specific dimension where the dimensions are selected to produce the desired PK profile.

Because the surface area to volume ratio of the micro-encapsulated drug is critical and because this ratio achieves favorable parameters over a narrow range, precision manufacture of the microencapsulated active drug is critical if a consistent PK profile with the desired characteristics is to be achieved.

A greater absolute change in surface area to volume ratio occurs from 1 to 30 microns sphere diameter as compared to the absolute change when using large particle sizes and holding the same total volume. The technology described above with reference to FIGS. 2 and 3 can be used to make micro encapsulated spheres in high yield and to tight specifications.

By surface area to volume ratio, what is meant is the surface area of the microsphere divided by the volume of the active drug “inner sphere” which is coated by the inactive ingredient forming the microsphere capsule. Note that when the capsule thickness is zero, there is only active drug in the microsphere.

Microspherical formulations of pharmaceutical preparations have been described before. For example, U.S. Pat. No. 6,153,129 describes the preparation of microspheres from emulsions of active and inactive ingredients. The result is formation of microspheres with a homogeneous blend of active drug and excipient in spheres of varying size. Further, U.S. Pat. No. 6,194,006 indicates that the size of spheres prepared in this way can be partly controlled during the spray drying process.

In order to produce microspheres for precision release as taught in this invention the microspheres are preferably of consistent size, enabling distinct spheres to be made 5 μm or 10 μm or 20 μm diameters with difference performance expected from each size. The importance of this kind of size consistency has not been previously appreciated.

The greatest absolute change in surface area to volume ratio occurs when the size of the diameter of the sphere changes from 1 μm to 20 μm. The specific surface area to volume values vary with capsule thickness.

### Heterogenous Particle Formulations

Formulations of the present invention are comprised of a plurality (2 or more) of groups of different types of different types of particles. A first group of spherical particles is present wherein each particle of the first group has a same diameter as other particles in the group with a margin of error in terms of particle diameter size of approximately ±10% or less. The formulation then includes a second group of spherical particles wherein each particle of the second group has the same diameter as the other particles in the second group with a margin of error of about ±10% or less. The particles within the first group are different from the particles within the second group and preferably have a difference in terms of the steady state blood levels which difference is sufficient to provide a longer steady state blood level than either of the groups by themselves. Preferably, the first group of particles and the second group of particles each comprise 100 or more particles, more preferably a 1,000 of more particles, and still more preferably 10,000 or more particles and may comprise 10⁵ to 10¹⁰ or more particles.

Although the heterogeneous formulations of the invention can be produced using particle formation technology of various types the technology as described above with respect to FIGS. 2 and 3 are preferred in that they produce very uniform sized and shaped particles. Further, the formulations may include particles which are solid spheres which may be produced using the technology as shown in FIG. 2. However, the preferred formulations of the invention include at least one group of particles wherein the particles are coated using the technology as shown within FIG. 3. Preferably, formulations of the invention include 3 or more groups of spherical particles wherein the particles within each group are the same and are different between the groups. Further, a preferred formulation will include at least some particles which are not coated e.g. a first group of particles with no coating and a relatively small particle size. Thus, the first group of particles will provide for substantially immediate dissolution and release of all of the compound or drug which is present in the particles. This causes the drug to quickly reach a therapeutic level in a patient's blood. The remaining groups of particles are coated and remain undissolved. When a known amount of time has passed the patient's metabolism will have removed from the patient's blood a sufficient amount of the drug added by the first group such that the concentration of the drug in the blood is beginning to decline the coating on the second group of particles will then dissolve so that the second group of particles now begins to add drug to the patient's blood thereby gradually increasing the concentration via the second group of particles at a rate substantially corresponding to the rate at which drug from the first group of particles is being metabolized out. This is shown within the graph of FIG. 5. The process can be repeated several times with several different groups of particles and three different groups of particles are shown within the graph of FIG. 6.

In a particularly preferred embodiment of the invention an opioid such as fentanyl is dissolved in a solvent which may be water, ethanol or a combination of water and ethanol. The solution of drug in the solvent is then coated with a polymer material which can be quickly cured by the addition of energy or evaporation as shown within FIG. 3. Thus, a group of particles is formed wherein the particles are comprised of a liquid center which liquid is comprised of a solution of drug and solvent in an outer core of polymer material which is substantially inert i.e. does not provide a pharmacological effect. Such particles are produced in a variety of different size ranges. Each size is used to produce a group of particles which, by itself, is sufficient to provide for therapeutic levels of a drug to a patient. When the coating dissolves the liquid within the spheres, which is a liquid drug (e.g. a drug in an aqueous solution) is immediately released. When the drug has metabolized to the point of beginning to drop below therapeutic levels the next group of particles with a thicker coating have dissolved to the point where the drug within these particles is released raising the patient's therapeutic level. By including a plurality of different groups it is possible to maintain the therapeutic level of the drug over a long period of time e.g. 1 day, several days (2 to 6 days) to 1 week, and even several weeks (2 to 3 weeks) to 1 month with injectable formulations.

Those skilled in the art will recognize that variability in terms of the rate at which the coating material dissolves can be changed by increasing the thickness of the coating and/or by changing the composition of the coating material as some materials will dissolve more quickly than others. Accordingly, the different groups of particles within the formulation may be particles which are all of the same size, but have different coating thicknesses. Alternatively, the particles may be all of the same size, and have the same coating thicknesses but have different coating compositions from one group to another wherein the composition of coating on one group of particles dissolves more rapidly than the coating composition on another group within the formulation.

### Agonists/Antagonists

The present invention can be used to administer a single drug or combinations of drugs. Further, different formulations can be co-administered to obtain desired effects. As an example of such it is pointed out that narcotics (agonists) cause certain undesirable side effects beyond pain relief. For example, morphine causes constipation which can be relieved by the administration of a narcotic antagonist provided the narcotic antagonist is of a type and delivered in an amount so as to not interrupt the analgesic properties of the narcotic. Various methods of using such combinations are disclosed in publications such as U.S. Pat. No. 6,277,384 directed to “opioid agonist/antagonist combinations.” Further, the use of particular antagonists to relieve constipation are disclosed in publications such as U.S. Pat. No. 4,176,186 entitled “Quaternary derivatives of noroxymorphone which relieves intestinal immobility.” It is desirable to use narcotic antagonists but which have the structures which substantially impede their ability to cross the blood-brain barrier. Accordingly, such narcotic antagonists do not substantially effect the pain relief characteristics of the narcotic but can block the adverse side effects such as constipation.

Formulations such as the formulations disclosed here can be created containing only narcotic antagonists which narcotic antagonists are characterized by not crossing the blood-brain barrier. Such formulations could be administered with conventional formulations in order to provide for a substantial reduction in side effects.

Although others have discussed methods of preventing the abuse of opioid drugs such as described within U.S. Pat. No. 6,228,863 entitled “Method of preventing abuse of opioid dosage forms” the present invention provides a unique approach to such. In accordance with the present invention the method involves having the dosage form administered by the physician in the physician's office. Other caregivers can, of course, also administer the dosage form in other locations such as a clinic or hospital. The dosage form should be designed so as to provide for a therapeutically effective amount of a drug to a patient over a period of three days or more, preferably one week or more, still more preferably two weeks or more. Because the dosage form is administered directly by the physician it can be more closely monitored as compared with self-administered drugs.

### Method of Reducing the Diversion of Narcotics

Those skilled in the art recognize that legal prescribed narcotics are, at times, diverted away from the patient for which they are intended. This problem exists when the patient has the narcotics in a form which can be used by others e.g. pills, patches, injectables, implants, etc. Implants can be taken out of a patient and patches can be removed and used by others. Pills can be taken by others if not already consumed by the patient. If the injectable is quick release then the patient will have drug which is not injected and which can be used by others.

The narcotics can be in substantially any form and be diverted particularly when the patient is sent home with multiple doses of the narcotic. Diversion can be reduced by the caregiver directly dosing the patient. However, such is impractical and prohibitively expensive. The present invention (1) addresses the diversion problem by putting the narcotics inside the patient where the narcotics can not be accessed by others and (2) addresses the practicality and expense issue by administering a controlled release injectable which provides continual pain relief to the patient for several days or even several weeks.

An aspect of the invention is a method of reducing the diversion of narcotics. The narcotic may be any narcotic including morphine and any opioid drug including fentanyl and sufentanyl. Including more potent drugs in the formulation makes it possible to administer drugs over a longer period of time without the need for administering an excessively large volume of formulation.

Initially the formulation is stored in two parts—drug particles and liquid. The dry particles are comprised of two or more groups and perhaps a plurality of groups of particles of different sizes. The particles may consist only of, or substantially only of drug. Alternatively, the particle may be comprised of excipient material which may encapsulate the drug. Particles and groups of particles are described in detail here. The liquid may be water, saline solution or any liquid suitable for injection.

The dry particles and liquid are mixed prior to injection to create a suspension. The suspension is injected in any desired manner which may be intramuscular or subcutaneous. The dry particles and liquid may be mixed by the caregiver and may be injected by the caregiver into the patient immediately after the suspension is created.

After the suspension is injected into the patient the patient may be released from care without risk of diversion of the injected drug. The injected particles will release drug to the patient over a period of time which may be days or weeks. During this period the drug may be continually released to the patient at a therapeutical level so as to provide a desired level of pain relief to the patient. Because the drug is inside the patient during this period, drug diversion is avoided during this period. The caregiver or the patient may monitor factors such as the level of pain relief obtained, the level of drug in the patient's circulatory system and/or various vital signs of the patient such as blood pressure, heart rate and respiratory rate. Dosing is generally started at a low level so that it can be increased as needed based on the monitored factors, e.g. the level of pain relief obtained.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Those skilled in the art will recognize that the technology described here can be provided to a number of different types of drugs and to heterogenous formulations of all different numbers of particle groups. However, here a specific example is described wherein the active drug is first included within particles which have no coating and thereafter are included within two additional groups of particles wherein the percent thickness of the spheres is varied.

| | Sphere Diameter | | |
|---|---|---|---|
| Capsule Thickness | 5 microns | 10 microns | 20 microns |
| 0 | S/V = 2.4 | S/V = 1.2 | S/V = 0.6 |
| 10% | S/V = 4.7 | S/V = 2.3 | S/V = 1.2 |
| 30% | S/V = 38 | S/V = 19 | S/V = 9.4 |

The surface area to volume ratio numbers in Table 6 must be taken in the context of the capsule thickness. Microspheres with a capsule thickness of zero are composed entirely of active drug; there is by definition no inactive ingredient forming a capsule layer. Therefore, even though a 10 μm microsphere with zero capsule thickness has the same surface area to volume ratio (1.2) as a 20 μm microsphere with a 10% capsule thickness, release of active drug from the 20 μm sphere will occur only after the outer layer has dissolved whereas active drug from the 10 μm sphere in this example will begin be released as soon as microsphere dissolution begins.

In addition, in the context of this invention, high surface area to volume values do not necessarily mean faster release of active drug into the circulation. This is because, for the case of non-zero capsule thickness microspheres, the outer material is an inactive ingredient.

By having a formulation in which a distinct capsule thickness is present in microspheres of a distinct size, a true programmable controlled release profile can be engineered by selecting (a) the capsule thickness and microsphere size and (b) by selecting in which proportions different populations of microspheres selected in (a) are combined into a dosage form.

For example, a slow release fentanyl or sufentanil formulation for subcutaneous injection could consist of ⅓ zero capsule thickness 5 μm microspheres for rapid release, ⅓ 10% capsule thickness 10 μm spheres for intermediate release and ⅓ 10% capsule thickness 20 μm microspheres for long term release as part of a single formulation. Because the capsule of inactive material must be largely dissolved before active drug release, this approach has the distinct advantage of minimizing the overlap of delivery by the various formulation components. This allows the aggregate PK profile of the formulation to be formed by superposition of the release profiles of the components of the formulation.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A formulation, comprising:
a first group of spherical particles wherein each particle of the first group has the same diameter as other particles in the first group with a margin of error of ±10% or less;
a second group of spherical particles wherein each particle of the second group has the same diameter as other particles in the second group with a margin of error of ±10% or less;
wherein the spherical particles of the first group and the spherical particles of the second group are comprised of a pharmaceutically active drug; and
wherein the first group and the second group each comprise 100 or more particles and further wherein particles of the first group dissolve at a rate which is faster than a rate at which the particles of the second group dissolve.

2. The formulation of claim 1, wherein the pharmaceutically active drug is a narcotic.

3. The formulation of claim 2, wherein the narcotic is an opioid drug selected from the group consisting of fentanyl and sufentanil.

4. The formulation of claim 1, further comprising:
a third group of spherical particles wherein each particle of the third group has the same diameter as other particles in the third group with a margin of error of ±20% or less;
wherein the spherical particles of the third group are comprised of an opioid drug selected from the group consisting of fentanyl and sufentanil; and
wherein the third group comprises 100 or more particles and further wherein particles of the third group dissolve at a rate different from a rate at which the particles of the first and second groups dissolve.

5. The formulation of claim 1, further comprising:
a plurality of additional groups of spherical particles wherein the particles of each additional group has the same diameter as other particles in that group with a margin of error of ±20% or less; and
wherein the spherical particles of each additional group are comprised of an opioid drug selected from the group consisting of fentanyl and sufentanil; and
wherein each additional group comprises 100 or more particles and further wherein particles of each additional group dissolve at a rate different from a rate at which the particles of other groups dissolve.

6. The formulation of claim 4,
wherein the second group of particles have 1,000 square centimeters or more of surface area more than the first group of particles; and
wherein the third group of particles have 2,000 square centimeters or more of surface area more than the second group of particles.

7. The formulation of claim 4,
wherein the second group of particles have 5,000 square centimeters or more of surface area more than the first group of particles; and
wherein the third group of particles have 10,000 square centimeters or more of surface area more than the second group of particles.

8. The formulation of claim 5, wherein each group of spherical particles is present in an injectable liquid carrier.

9. The formulation of claim 8, wherein the particles of each group have a specific gravity relative to the liquid carrier such that the particles of each group are suspended in the carrier, creating a uniform suspension with the particles uniformly dispersed in the liquid carrier.

10. The formulation of claim 5, wherein the particles of each group dissolve at a rate per unit of time which is different from a rate of dissolution of any other of the groups of particles by an amount of about 10% or more.

11. The formulation of claim 8, wherein the particles of each group dissolve at a rate per unit of time which is different from a rate of dissolution of any other of the groups of particles by an amount of about 25% or more.

12. The formulation of claim 8, wherein the pharmaceutically active drug is a narcotic antagonist.

13. The formulation of claim 5, wherein the spherical particles of the second group are coated particles wherein the coating is comprised of a pharmaceutically acceptable carrier.

14. The formulation of claim 5, wherein the spherical particles in each group have a diameter in a range of from about 40 micrometers to about 2 micrometers.

15. The formulation of claim 5, wherein the spherical particles in each group have a diameter in a range of from about 30 micrometers to about 4 micrometers.

16. A formulation, comprising:
a first group of spherical coated particles wherein each particle of the first group has an outer diameter substantially the same as other particles in the first group with a margin of error of ±20% or less and wherein the particles have a flowable liquid center surrounded by an outer coating;
a second group of coated spherical particles wherein each particle of the second group has substantially the same diameter as other particles in the second group with a margin of error of ±20% or less and wherein the coated spherical particles of the second group are comprised of a liquid flowable core surrounded by an outer coating;
wherein the flowable liquid center of the spherical particles of the first group and the flowable liquid center of the spherical particles of the second group are comprised of a solution of a pharmaceutically active drug; and
wherein upon administration to a biological system the particles of the first group release the liquid core at a different time from the time at which the particles of the second group release the inner core.

17. The formulation of claim 16, further comprising:
a third group of coated spherical particles wherein each particle of the third group has the same diameter as other particles in the third group with a margin of error of 120% or less and wherein the coated spherical particles of the third group are comprised of a liquid flowable core surrounded by an outer coating;
wherein the flowable liquid center of the spherical particles of the third group are comprised of a solution of a pharmaceutically active drug; and
wherein upon administration to a biological system the particles of the third group release the liquid core at a different time from particles of the first and second groups.

18. The formulation of claim 16, further comprising:
a plurality of additional groups of coated spherical particles wherein the particles of each additional group have the same diameter as other particles in that group with a margin of error of ±20% or less and wherein the coated spherical particles of each additional group are comprised of a liquid flowable core surrounded by an outer coating; and
wherein the flowable liquid centers of the spherical particles of each additional group are comprised of a solution of a pharmaceutically active drug; and
wherein upon administration to a biological system the particles of each group releases the liquid core at a different time from other groups.

19. The formulation of claims 16, wherein the coating also surrounds a quantity of gas.

20. The formulation of claims 16, further comprising:
a liquid carrier surrounding the particles of the first group and the particles of the second group and the particles of any additional groups wherein the liquid carrier is a pharmaceutically acceptable injectable carrier.

21. The formulation of claim 18, wherein the pharmaceutically active drug is a narcotic.

22. The formulation of claim 21, wherein the narcotic is an opioid drug selected from the group consisting of fentanyl and sufentanil.

23. The formulation of claim 17, wherein each of the groups of coated spherical particles are suspended in the liquid carrier, and wherein the particles of each group having coatings which dissolve in a biological system and release the flowable liquid center at a different time relative to any other group of particles in the formulation and wherein the difference in time between each group is 10% or more of the total time for all groups to release the flowable liquid center.

24. The formulation of claim 17, wherein the coated spherical particles are produced by a process, comprising the steps of:
forcing a liquid formulation comprising a pharmaceutically active drug through a channel of a first feeding source in a manner which causes a stream of the liquid drug to be expelled from a first exit opening at a first velocity;
forcing a liquid comprising a coating material through a second channel concentrically positioned around the first channel in a manner which causes a stream of the liquid coating material to be expelled from a second exit opening at a velocity which is substantially the same as the first velocity whereby the stream of coating material is concentrically positioned around the stream of liquid drug;
forcing a gas through a pressure chamber surrounding the exit openings of the concentrically positioned first and second channels in a manner which causes the gas to exit the pressure chamber from an exit orifice positioned downstream of the concentrically positioned streams of liquid drug and coating material;
wherein the density of the liquid formulation comprising the pharmaceutically active drug is substantially the same as the density of the liquid comprising the coating material, and the gas focuses the concentrically positioned streams to a stable unified jet which flows out of the chamber exit orifice and breaks up into coated particles of the pharmaceutically active drug coated with the coating material.

25. The method of claim 24, wherein the stable unified jet comprises a diameter d, at a given point A in the stream characterized by the formula:
$${d}_{j} ≅ {( \frac{8 {ρ}_{1}}{{π}^{2} Δ {P}_{g}} )}^{1 / 4} {Q}^{1 / 2}$$
wherein dⱼ is the diameter of the stable unified jet, ≅indicates approximately equally to where an acceptable margin of error is ±10%, ρ₁ is the average density of the liquid of the jet and ΔP_{g} is change in gas pressure of gas surrounding the stream at a given point A and Q is the total flow rate of the stable unified jet.

26. The method of claim 25, wherein dⱼ is a diameter in a range of about 1 micron to about 1 mm.

27. The method of claim 25,
wherein the stable unified jet has a length in a range of from about 1 micron to about 50 mm;
wherein the stable unified jet is maintained, at least in part, by tangential viscous stresses exerted by the gas on a surface of the jet in an axial direction of the jet; and
wherein the stable unified jet is further characterized by a slightly parabolic axial velocity profile.

28. The method of claim 25, wherein the particles of pharmaceutically active drug coated with coating material are characterized by having the same diameter with a deviation in diameter from one particle to another in a range of from about ±3% or less.

29. The method of claim 28, wherein the deviation in diameter from one particle to another is in a range of from about ±1% or less.

30. The method of claim 25, wherein a coated particle of the first group has a diameter in a range of about 0.1 micron to about 100 microns and other particles of the first group have the same diameter as the given particle with a deviation of about ±3% or less; and
wherein ΔP=P₀-P₁, the difference in pressure through the chamber exit orifice, is equal to or less than twenty times the surface tension of the liquid comprising the coating material with the gas, divided by the radius of the stable unified jet.

31. The formulation of claim 17,
wherein the second group of particles have 1,000 square centimeters or more of surface area more than the first group of particles; and
wherein the third group of particles have 2,000 square centimeters or more of surface area more than the second group of particles.

32. The formulation of claim 17,
wherein the second group of particles have 5,000 square centimeters or more of surface area more than the first group of particles; and
wherein the third group of particles have 10,000 square centimeters or more of surface area more than the second group of particles.

33. A method of reducing diversion of a narcotic, comprising the steps of:
injecting suspension into a patient wherein the suspension is comprised of a plurality of groups of particles;
allowing the particles to release narcotic drug to the patient over a period of time in a range of from about three days to about 30 days.

34. The method of claim 33, further comprising:
mixing a liquid and a plurality of groups of particles to create the suspension.

35. The method of claim 33, wherein the injection is intramuscular.

36. The method of claim 33, wherein the particles comprise:
a first group of spherical particles wherein each particle of the first group has the same diameter as other particles in the first group with a margin of error of ±20% or less;
a second group of spherical particles wherein each particle of the second group has the same diameter as other particles in the second group with a margin of error of ±20% or less;
wherein the spherical particles of the first group and the spherical particles of the second group are comprised of a narcotic; and
wherein the first group and the second group each comprise 100 or more particles and further wherein particles of the first group dissolve at a rate which is faster than a rate at which the particles of the second group dissolve.
